(19) United States
(12) Patent Application Publication
(10) Pub. No.: US 2005/0049291 A1
(43) Pub. Date: Mar. 3, 2005

(54) Process for the preparation of pharmaceutical compositions for topical delivery of cyclooxygenase-2-enzyme inhibitors

(76) Inventors: Mukesh Kumar, Distt.-Ludhiana (IN); Ajay Kumar Singla, Chandigarh (IN); Shirumalla Raj Kumar, New Delhi (IN); Vinod Kumar Arora, New Delhi (IN); Rajiy Malik, Wien (AT)

Correspondence Address: RANBAXY INC., 600 COLLEGE ROAD EAST, SUITE 2100, PRINCETON, NJ, 08540, US

(21) Appl. No.: 10/493,457

(22) PCT Filed: Oct. 23, 2002
(86) PCT No.: PCT/IB02/04401

(30) Foreign Application Priority Data
Oct. 23, 2001 (IN) 1073/DEL/01

(51) Int. Cl. A61K 31/415; A61K 9/14

(52) U.S. Cl. 514/406; 514/471; 424/486; 424/488

(57) ABSTRACT

A present invention relates to a pharmaceutical composition for topical delivery comprising a pharmaceutically effective amount of drug(s), that acts selectively as a cyclooxygenase-2 enzyme inhibitor. The composition provides better percutaneous absorption and enhanced efficacy.

### FIELD OF THE INVENTION

The present invention relates to a pharmaceutical composition for topical delivery comprising a pharmaceutically effective amount of drug(s), that acts selectively as a cyclooxygenase-2 enzyme inhibitor. The composition provides better percutaneous absorption and enhanced efficacy.

### BACKGROUND OF THE INVENTION

Due to availability of large surface area, easy accessibility, application dynamics and the non-invasive nature of the therapy, topical administration of drugs has long been considered a promising route of drug delivery whether the bioavailability desired is systemic, dermal, regional or localized. This mode of drug delivery provides many advantages over customarily used routes of administration. It bypasses the portal circulation and thereby the hepatic first-pass metabolism, avoids the variable systemic absorption and metabolism and also, potentially reduces gastro-intestinal irritation associated with oral administration. Further, it avoids the risks and patient non-compliance associated with parenteral treatment. Topical route offers continuity of drug administration, permits use of therapeutic agents with short biological half-lives, provides treatment of cutaneous manifestations of diseases usually treated systemically, delivers medication directly into the systemic circulation and foster ease of use and total patient compliance.

Host of patents have been granted pertaining to topical compositions of drugs. By way of example, U.S. Pat. No. 5,093,133 discloses a hydroalcoholic gel of pH 3.5-6.0 consisting essentially about 1-15% substantially pure S-ibuprofen, 0-20% of propylene glycol, about 40-60% alcohol, about 2-5% of a gelling agent selected from the group consisting of hydroxypropyl cellulose and polyacrylic acid polymers and about 0.25-2% of triethanolamine to adjust the pH. The rate of delivery of ibuprofen from such a system is allegedly pH dependent. It is believed that such a topical system wherein such high concentration of alcohol is used, repeated application could cause unfavorable conditions.

U.S. Pat. No. 5,976,566 describes the use of 1,3-dioxane and 1,3-dioxolane derivatives or acetal as skin penetration enhancers for NSAIDs. It discloses a substantially neutral ibuprofen containing alcoholic or aqueous alcoholic composition which comprises a skin penetration enhancing effective amount in the range of from about 4-15% of a C₇ to C₁₄-hydrocarbyl substituted 1,3 dioxolane, 1,3-dioxane or acetal, about 0-18% of glycol, at least about 40% of volatile alcohol, base to provide a pH in the range of from about 6.5 to about 8 and, optionally, gelling agent effective to thicken the composition to avoid or minimize run-off when applied to the skin. The penetration enhancers used therein are unstable at lower pH. The invention is particularly adapted only for NSAIDs in substantially neutral salt form (pH 6-8) which allegedly makes the gel formulation stable.

U.S. Pat. No. 4,602,040 describes non-aqueous clear gel and topical cream composition of meclofenamic acid. Essentially, the patent discloses a clear gel formulation of meclofenamic acid in a cosolvent system of a polyethylene glycol ester, water soluble lanolin oil, an alcohol and a thickening agent and a cream formulation which is homogenized emulsion of polyethylene glycol ester, glyceryl or propylene glycol ester, triglyceride and mineral oil.

An anti-inflammatory analgesic gel composition, as disclosed through U.S. Pat. No. 4,393,076, comprises ketoprofen as the active ingredient, a glycol, lower alcohol, water and/or a mixture of a lower alcohol with water, a gel forming agent and optionally, a solubilizing agent and/or nonionic surface active agents as penetration enhancers.

U.S. Pat. No. 5,807,568 describes enhanced delivery of flurbiprofen through topical compositions comprising 0.5 to 10% of active, about 10-80% of a lower alcohol, about 0-25% of a glycol, about 0-5% of a gelling agent, an amount of a pH adjusting agent sufficient to adjust the pH of the composition to a range of from about 2 to less than 4.5 and water in an amount sufficient to make up the composition.

As mentioned above, several pharmaceutical compositions are described in literature for topical application of nonsteroidal anti-inflammatory drugs (NSAIDs) which are known to be the most commonly prescribed group of drugs worldwide for analgesic, antipyretic and anti-inflammatory effects. Adverse reactions, mostly associated with gastrointestinal disturbances such as acidity, ulceration, hepatic and nephric disorders etc. have been reported with repeated oral NSAID therapy. Hitherto, topical application is one of the preferred alternative routes of administration. Direct application to inflammed joints results in appreciably lower systemic blood levels, reduced gastrolesivity and thereby better tolerance.

Further, NSAIDs are known to act through inhibition of cyclooxygenase and lipoxygenase pathway of arachidonic acid metabolism. The cyclooxygenase (COX) enzyme catalyses the first step in the conversion of arachidonic acid to prostanoids (prostaglandins and thromboxanes). The central mechanism leading to the therapeutic effects of NSAIDs is through the blockade of prostaglandin synthesis resulting from inhibition of cyclooxygenase enzyme. The gastrointestinal adverse effects of these drugs are also largely attributable to cyclooxygenase inhibition. Recent research has revealed that this enzyme exists in 2-isoforms, COX-1 and COX-2. It is proposed that inhibition of COX-1 results in their shared adverse effects, whilst COX-2 being the primary isoform available at the sites of inflammation, its inhibition accounts for the therapeutic benefits of NSAIDs.

Fuelled by this hypothesis, much of the recent research has focussed upon efficacious methods for development of drug delivery of COX-2 enzyme inhibitors to treat inflammation associated maladies.

Our published International Patent Application No. WO 02/17923 describes a pharmaceutical composition for topical delivery of cyclooxygenase-2-enzyme inhibitor. The pharmaceutical composition constitutes a topical delivery system, comprising cycloxygenase-2 enzyme inhibitor, a gelling agent, a solubilizing agent and, optionally, a pH modifying agent and/or other pharmaceutically acceptable adjuvants.

It has now been discovered that, micronized drug or drug in submicron size range, when incorporated to the pharmaceutical compositions described in International Patent Application No. WO 02/17923 results in readier dissolution, increased rate of absorption, enhanced percutaneous absorption and hence better topical efficacy.

The physico-chemical attributes and bio-pharmacological characteristics accounts for the formulation of a bioavailable pharmaceutical composition. The wettability and hence dissolution properties of a biologically active substance or drug precursor greatly influence the bioavailability. Poor percutaneous absorption is a significant problem encountered in the development of topical preparations, containing drugs that are poorly soluble in water such as cyclooxygenase-2 enzyme inhibitors. It has been observed that by increasing the surface area of a particulate drug, such as by decreasing the particle size, the rate of dissolution of the drug is increased thereby resulting in better permeability and percutaneous efficacy.

### SUMMARY OF THE INVENTION

In accordance with one aspect of the present invention, there is provided a pharmaceutical composition for topical delivery of COX-2 enzyme inhibitors that provides enhanced percutaneous bioavailability and better efficacy.

In accordance with a second aspect of the present invention, there is provided a pharmaceutical composition that results in rapid penetration of COX-2 enzyme inhibitor dissolved or suspended therein effecting significant protection of the carrageenan induced paw edema formation at an effective dose (ED₅₀) of not more than 15 mg/kg.

In accordance with a third aspect of the present invention, there is provided a composition that provides enhanced skin penetration and achieve therapeutic levels of the COX-2 enzyme inhibitors in target internal tissues.

In accordance with another aspect of the present invention, there is provided a composition, with low dermal irritation and skin sensitization.

According to yet another aspect of the present invention, there is provided a composition that has good stability and good cosmetic characteristics.

Accordingly, the present invention accentuates a pharmaceutical composition containing as drug a cyclooxygenase-2 enzyme inhibitor for topical application, which effects readier solubility of the active ingredient and which effectively transports the active through the barrier of the stratum corneum, and to the use thereof. As embodied and fully disclosed herein, the present invention describes a pharmaceutical composition for topical administration comprising a pharmaceutically effective amount of micronized drug(s) that acts selectively as a cyclooxygenase-2 enzyme inhibitor, a gelling agent, and a solubilizing agent.

The present invention also comprehends a pharmaceutical composition incorporating COX-2 inhibitor in the carrier base, a pH modifying agent and pharmaceutical adjuvants such as penetration enhancers, humectants and/or moisturizers, preservatives, opacifiers, fragrances, color additives, counter-irritants, and the like.

The pharmaceutical compositions of the invention are intended for topical, non-invasive application to the skin, particularly to the region where the COX-2 enzyme inhibitor is intended to exert its pharmacological activity, usually to a region of inflammation, injury or pain, to the muscles or joints, or other forms of cutaneous disorders or distruptions characterized by skin inflammation and/or hyperproliferative activity in the epidermis of skin.

According to the present invention, the pharmaceutical compositions are such that it provides release of at least one therapeutic agent or drug. The drug may be pharmacologically active itself or may be converted into the active form by biotransformation in the body. The combination of drugs that are typically administered together may be included as the drug component. However, in embodiments wherein such a combination is used at least one of such drug acts selectively as a cyclooxygenase-2 enzyme inhibitor.

Illustrative examples of the COX-2 enzyme inhibitors that are advantageously administered by the pharmaceutical compositions of this invention include specific inhibitors such as celecoxib, valdecoxib, rofecoxib, varecoxib, parecoxib, and the like or preferential inhibitors such as meloxicam, nimesulide, etodolac, and the like.

In a particular preferred embodiment of the present invention, the composition contains celecoxib or rofecoxib as the drug.

The drug itself or its pharmacologically active salt or ester can be used in the present invention. The amount of drug suitable for the present invention is that which is typically administered for a given period of time. This includes a pharmaceutically effective amount of the drug which is an amount high enough to significantly positively modify the condition to be treated, but low enough to avoid serious side effects (at a reasonable benefit/risk ratio), within the scope of sound medical judgement. The precise amount of drug will vary with the specific drug, the ability of the composition to penetrate the drug through the skin, the amount of the composition to be applied, the particular condition being treated, the severity of the condition, the duration of the treatment, the nature of concurrent therapy, the age and physical condition of the patient being treated, and the like factors. Accordingly, the drug dissolved or dispersed therein, may be present in amount ranging from a pharmaceutically effective amount from about 0.1% to about 25% by weight of the total weight of the composition.

The present invention is directed to a topical pharmaceutical composition exhibiting enhanced absorption and better efficacy of cyclooxygenase-2 enzyme inhibitor which is attained through incorporation of micronized and submicronized drug particles into a vehicle suitable for topical application to the skin. As used herein, the micronized or submicron size range refers to a particle size as measured by conventional size measuring techniques well known to those skilled in the art, such as sedimentation field flow fractionation, photon correlation spectroscopy, disk centrifugation or lazer diffraction technique. The fine particle size of the COX-2 enzyme inhibitor is critical to the present invention wherein the distribution of particle size in the micron and preferably in the submicron size range. Accordingly, the particle size of the drug is such that the mean particle size is less than about 30 microns, preferably less than about 5 microns and more preferably less than about 0.9 microns.

According to the present invention, the micronized drug particles have a wetting agent adsorbed onto the surface thereof. The wetting agent, herein, refers to any substance known in the art of formulation development that facilitates increase in the wettability and thus dissolution of the drug. By adsorbed, it is meant that the drug particles are closely associated with the wetting agent as a physical adherence but do not chemically interact with the drug. Such an interaction is undesirable as it could result in altering the function of the drug, itself. Furthermore, the individually adsorbed molecules of the wetting agent are essentially free of intermolecular cross-linkages. Representative examples of wetting agents include gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, e.g. macrogol ethers such as cetomacrogol 1000, polyoxyethylene castor oil derivatives, polyoxyethylene sorbitan fatty acid esters, e.g. the commercially available Tweens, polyethylene glycols, polyoxyethylene stearates, colloidal silicon dioxide, phosphates, sodium dodecylsulfate, sodium laurylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminium silicate, triethanolamine, polyvinyl alcohol and polyvinylpyrrolidone (PVP). Most of these excipients are described in detail in the Handbook of Pharmaceutical Excipients, published jointly by the American Pharmaceutical Association and the Pharmaceutical Society of Great Britain, the Pharmaceutical Press, 1986, the disclosure of which is herein incorporated by reference in its entirety.

The concentration of the wetting agent may vary from about 0.1 to about 90% and preferably between about 1 to about 75% by weight based on the total combined weight of the drug and the wetting agent.

According to the present invention, the composition contains an agent which provides the desired integral gel structure to the composition. The choice of gelling agents to be used is considered to be within the purview of one skilled in the art, provided they are compatible with the drug, solubilizing agents and other adjuvants.

The gelling agents preferred for the present invention include inorganic and organic macromolecules capable of forming gel structure. They may be of the hydrophilic or the hydrophobic type or pH dependent or pH independent in nature. Examples of gelling agents suitable for this invention include the agents well known in the pharmaceutical art for their gelling properties and may be selected from the group comprising cellulose ethers such as hydroxypropyl cellulose, hydroxyethyl cellulose, hydroxyethyl methylcellulose, methylcellulose, hydroxypropyl ethylcellulose, hydroxypropyl methylcellulose, carboxymethyl cellulose, sodium carboxymethyl cellulose, hydroxycellulose, and the like; vinyl alcohols such as Polyviol or Moviol, and the like; vinyl pyrrolidones such as Kollidon or Plasdone, and the like; natural gums such as karaya gum, locust bean gum, guar gum, gelan gum, xanthan gum, gum arabic, tragacanth, carrageenan, pectin, agar, alginic acid, sodium alginate, and the like; acrylic polymers such as methacrylates such as available as Eudragit and polyacrylates such as available under the brandname Carbopol; polyoxyethylene-polyoxypropylene copolymers (Poloxamer) such as available as Lutrol, and the like.

According to the present invention, the composition contains polyacrylate or poloxamer as the gelling agent.

The requisite amount of gelling agent used in this invention is an amount needed to obtain a gel formulation of desirable consistency that allows for easy application to the skin. A low concentration of gelling agent makes the formulation loose or fluid which runs on application, while higher concentration results in stiff formulation that are not easily spreadable. The gelling agents may be present from about 0.3% to about 40%, preferably from about 0.5% to about 30% by weight of the total weight of the composition.

According to the present invention, the pharmaceutical composition contains solubilizing agents which aids in the solubility and better penetration of the drug through skin. The solubilizing agents may be volatile, or non-volatile in nature or a combination thereof.

The compositions of the invention may contain a volatile solubilizing agent that includes especially lower alkanols having preferably 2 or 4 carbon atoms such as ethanol, denatured ethanol (commercially available as SDA-40), propanol, isopropanol, butanol and mixtures thereof. Other pharmaceutically acceptable alcohols may also be used in this invention.

According to the present invention, the compositions may comprise non-volatile solubilizing agent. Examples of non-volatile solubilizing agents that may be used in the present invention include glycols and derivatives thereof such as butylene glycol, propylene glycol, polypropylene glycol, polyethylene gycol, hexylene glycol, polyethylene glycol dodecyl ether, diethylene glycol monoethyl ether (available commercially as Transcutol), polyethylene glycol-8 glyceryl caprylate (commercially available as Labrasol), propylene glycol monocaprylate (commercially available as Capryol 90), and the like; polysorbates such as available as Tween 20, Tween 40, Tween 60, Tween 80, and the like; Sorbitan esters such as sorbitan monolaurate (Span 20), sorbitan monopalmitate (Span 40), sorbitan monostearate (Span 60), sorbitan trioleate (Span 85), and the like; polyoxyl oil derivatives such as polyoxyl 60 hydrogenated castor oil, polyoxyl castor oil, polyoxyl 35 castor oil, polyoxyl 40 hydrogenated castor oil (available as Cremophor RH40), and the like. Other pharmaceutically acceptable solubilizing agents such as dimethyl sulfoxide, dimethyl formamide, benzyl alcohol, and the like may also be used. These solubilizing agents may be used alone or in a mixture of at least two or more.

The total amount of the solubilizing agent used, depends on the factors such as amount of COX-2 inhibitor, type of COX-2 inhibitor, amount and nature of the gelling agent, and the like. However, the composition of the invention may contain solubilizing agents in an amount from about 2% to about 60%, preferably from about 5% to about 50% and more preferably from about 10% to about 40% by weight of the total weight of the composition.

According to the present invention, the pharmaceutical composition contains combination of ethanol, polyethylene glycol-8 glyceryl caprylate, polyethylene glycol and propylene glycol as the solubilizing agents.

These compositions containing alcohol are of great utility in solubilizing active ingredients which are poorly soluble in glycol but highly soluble in alcohol. Moreover, the alcohol contained in the composition exerts a bactericidal and bacteriostatic effects on skin areas to which the compositions are applied, and provides a cooling counter-balance to the glycol solubilizing agents which may sometimes create a warming sensation when applied to the skin. The solubilizing agents disclosed herewith provide unique advantages. Such a system provide stable non-irritating composition of a wide variety of drugs and aids in penetration of COX-2 enzyme inhibitors with even high molecular weights through the skin.

The interplay of alcohol and glycols as a solubilizing agent improves solubility of polar drugs and those that are primarily sparingly soluble in water. In addition, such a combination promotes improved resorbability of the COX-2 enzyme inhibitor. Further, such a combination improves spreadability and aestethics of the pharmaceutical composition. It minimizes any congealing or balling up or drying of the composition when it is rubbed on the skin. Furthermore, polyethylene glycol-8 glyceryl caprylate being a surfactant acts as a permeation enhancer and hence improves penetration of the COX-2 enzyme inhibitor. Also, such a combination gives better consistency, as ethanol or polyethylene glycol-8 glyceryl caprylate or polyethylene glycol alone results in a composition with high fluidity, whilst propylene glycol alone results in a tacky composition, which does not spread uniformly.

According to the present invention, the compositions may also comprise a pH modifying agent. The present invention is directed to a pharmaceutical composition exhibiting an optimal flux or diffusion for the topical delivery of COX-2 enzyme inhibitors. It is well known to the one skilled in art that composition at optimal pH maximizes the flux i.e. the rate of delivery of the drug through skin. Further, most gelling agents usable in accordance with the present invention are highly acidic which drop the pH below the desirable range. Furthermore, certain gelling agents in accordance with the present invention form integral gel structure only at near neutral pH. Carboxyvinyl polymers is one such example. These are hydrophillic polymers that are prepared by polymerizing monomers principally consisting of acrylic acid. Due to the presence of free carboxylic acid residues, an aqueous solution of this polymer is acidic in nature. Neutralization of this solution cross-links and gelatinizes the polymer to form a viscous integral structure of desired viscosity.

Accordingly, any well known and pharmacologically safe inorganic or organic basic compounds can be used for modifying the pH. Examples of inorganic basic salts that may be used in the present invention include ammonium hydroxide, alkali metal salts, alkaline earth metal salts such as magnesium oxide, magnesium hydroxide, calcium hydroxide, sodium hydroxide, potassium hydroxide, lithium hydroxide, aluminium hydroxide, potassium carbonate, sodium bicarbonate, and the like. The examples of organic basic salts that may be used in the present invention include alkanolamines such as methanolamine, ethanolamine, propanolamine, butanolamine, dimethanolamine diethanolamine, dipropanolamine, dibutanolamine, diisopropanolamine, trimethanolamine triethanolamine, tripropanolamine, diisopropanolamine, tributanolamine, aminomethylpropanol, N-methyl glucamine, tetrahydroxypropyl ethylene diamine, and the like; alkylamines such as methylamine, ethylamine, propylamine, butylamine, diethylamine, dipropylamine, isopropylamine, and the like.

According to the present invention, the pharmaceutical composition contains triethanolamine as the pH modifying agent.

For any particular composition, the drug and likewise the other ingredients may be selected to achieve the desired release profile and the extent of penetration. The optimum pH may then be determined and will depend on factors such as nature of COX-2 enzyme inhibitor, gelling agent, degree of flux required, and the like. However, the pH of the pharmaceutical composition according to the present invention may be between 2.0 and 8.0, and preferably between 4.0 and 7.0.

Optionally, there may also be incorporated into the pharmaceutical composition of the present invention other conventional pharmaceutically acceptable adjuvants known in the art of formulation development such as penetration enhancers, humectants and/or moisturizers, preservatives, opacifiers, fragrances, color additives, counter-irritants and the like. The adjuvants selected should be such that there is no interaction which would substantially reduce the pharmaceutical efficacy of the composition of the present invention. Pharmaceutical adjuvants used must be of high purity and low toxicity to render them suitable for administration.

The composition of the invention may further comprise penetration enhancers for improved transepidermal or percutaneous delivery of drug. The penetration enhancers suitable for the present invention include terpenes, terpene alcohols, essential oils, surfactants, and the like. Some such examples include d-limonene, terpinen-4-ol, menthone, 1,8-cineole, 1-pinene, α-terpineol, carveol, carvone, pulegone, eucalyptol, peppermint oil, sorbitan esters, polysorbates, sodium lauryl sulphate, and the like.

The pharmaceutical compositions in accordance with the present invention may also contain one or more humectants and/or moisturizers. These may include polyhydroxy alcohols such as sorbitol, glycerin, hexanetriol, butanediol, mannitol, glucose, ethylene glycol, propylene glycol, and the like.

Preservatives such as methylparaben, propylparaben, phenoxyethanol, benzyl alcohol, bromopol, chlorocresol, thiomersal, benzalkonium chloride, and the like may be added to the compositions to inhibit microbial activity.

Opacifiers, such as behenic acid, glycol distearate, lard glycerides, polyethylene glycol esters, and the like; fragrances such as amyl salicylate, p-anisaldehyde, anisylalcohol, peppermint oil, wintergreen oil, and the like; colour additives such as quinoline yellow, and the like; counter-irritants such as methyl salicylate, menthol and the like; and other pharmaceutical adjuvants may be added to the compositions of the invention.

According to the present invention, the topical compositions provide significant protection of the carrageenan induced paw edema formation in rats which is a well documented and practiced animal model for determination of effective dose (ED₅₀) of anti-inflammatory drugs. Accordingly, the compositions provide protection to the carrageenan induced paw edema formation at an effective dose of not more than 15 mg/kg and preferably at a dose of not more than 11 mg/kg.

Preferably, the composition of the present invention may have a viscosity of within the range of about 25,000 to 4,00,000 centipoises (cps), preferably between about 100,000 to 3,00,000 cps, and even more preferably between about 800,000 to 2.0 million cps, when measured using a Brookfield type RVT series viscometer with helipath stand at ambient temperature (20° C.) and with a 0.5 inch helipath and T-spindle (size “C”) rotating at 2.5 RPM in a sample size ranging from 90-100 grams.

The compositions hereof have good stability. They do not show any substantial changes in viscosity at high temperatures or crystallization at low temperatures. Moreover, they adhere well to the skin and spread readily. Further, they do not impart a sticky feeling and dry easily.

Cox-2 enzyme inhibitors is subjected to size reduction upto submicron range using milling apparatus whereby it is reduced to a very fine powder due to attrition of the particles by collisions between particles and between particles and machine surfaces under the direct, high velocity collision force. For the purpose, any suitable apparatus that utilizes the crushing action under force between two surfaces, may be employed. Accordingly, the milling machines that work on substantially the same principle may be used. Examples of such milling machines include various makes of rotating ball mill, vibrational ball mill, attrition mill, automatic mortar mill, roller mill, gyratory mill, comminuting mill, media mills such as sand mill and bead mill, and the like. The particle size reduction may be advantageously carried out in a media mill. The principle of operation of this mill is one of the high velocity impact between rapidly moving beads and the powdered drug particles in a liquid medium (premix). It is preferred that the wetting agent be incorporated in the premix. As the beads hit, premix containing the drug, the wetting agent gets loaded onto the drug due to the impact generated by the same which warrant better wettability and facilitate absorption through skin. The period of loading, that is, the attrition time, may vary depending upon the particular mechanical means and processing conditions selected such as size of the mill, the speed of the rotation of grinding beads, type of feed material and quantity of feed material. The effects of these variables are well known in the art and the invention may be worked over a range of these variables.

The in vitro release profiles were characterized using modified Franz diffusion cells consisting of two compartments, a donor and a receptor, separated by a cellulose acetate nitrate (0.45μ) membrane on which a thin layer of test product was uniformly spread, whilst isopropyl alcohol and water mixture was used as a medium to maintain the sink conditions in the receptor compartment. The cellulose acetate nitrate membrane hinders the penetrant as it diffuses through its channels and the transport process correlates at best with molecular permeation across porous capillary endothelium. However, the transport mechanism is diffusion or passage through macroscopic ducts filled with solvent. All studies were conducted at 32° C.

### DETAILED DESCRIPTION OF THE INVENTION

The following examples further illustrate this invention, and are not to be construed as limiting the same but read in conjunction with the description above, provide further understanding of the present invention and an outline of the process for preparing the compositions of the invention.

### EXAMPLE 1

This example illustrates the preparation of a pharmaceutical composition of celecoxib. It depicts the effect of different concentrations of ethanol on the in vitro release profile of the drug. The pharmaceutical composition is given in Table 1.

TABLE 1

| Ingredients | Quantity (% w/w) |
|---|---|
| Celecoxib | 5.0 |
| Carboxypolymethylene (Carbopol 940) | 1.2 |
| Polyethylene glycol (PEG-400) | 12.5 |
| Propylene Glycol | 5.0 |
| Polyethylene glycol-8 glyceryl Caprylate | 5.0 |
| (Labrasol) | |
| Ethanol | 7.5 |
| Polyoxyl 40 Hydrogenated Castor | 0.5 |
| Oil (Cremophor RH40) | |
| Triethanolamine | 1.0 |
| Phenoxyethanol | 1.0 |
| Fragrance (Oil of lemon lime) | 0.34 |
| Purified water | to 100 |

Dissolved Polyoxyl 40 Hydrogenated Castor Oil in warm purified water and dispersed celecoxib (micronized, mean particle size, 14.68μ) under continuous stirring. Added, under stirring, polyethylene glycol, propylene glycol, polyethylene glycol-8 glyceryl caprylate and phenoxyethanol. Carboxyvinyl polymer was further dispersed in the resultant dispersion following which ethanol and fragrance was also added. Triethanolamine, dissolved in a portion of water (about 50 ml) was then added which initiated viscous structure formation. The weight was made upto 500 g with purified water and the resultant mixture was thoroughly mixed until wholly been made homogenous to obtain an anti-inflammatory analgesic topical composition. The resultant composition had a pH of 6.68 and a viscosity of 1,78,000 cps.

Alternatively, a gel formulation was prepared identically having 30% w/w alcohol content. The resultant composition had a pH of 6.46 and a viscosity of 1,52,000 cps.

Alternatively still, the pharmaceutical composition was prepared using celecoxib having a mean particle size in submicron size range. Sodium laurylsulfate (0.05% w/w) and polyvinyl pyrrolidone (Povidone K30, 1.0 w/w) were dissolved in warm purified water. Celecoxib (8% w/w) was dispersed in this solution under continuous stirring and the resultant suspension was milled in a bead mill (Dyno mill) to attain the mean particle size of 4.0μ. The topical gel formulation was prepared identically using milled celecoxib by the procedure described above. The resultant composition had a pH of 6.72 and a viscosity of 1,65,000 cps.

The two compositions (micronized and submicronized) were studied for in vitro release profile using modified Franz diffusion cells. The samples of the receptor media (IPA:Water::55:45) were analyzed for celecoxib content at regular intervals, spectrophotometrically. The results given in Table 2 shows the release profiles of the formulations under discussion (Table 1) as per the present innovation and a gel formulation prepared identically having higher concentration of the volatile solubilizing agent (alcohol, 30% w/w).

TABLE 2

Flux (μg/ml/cm²)

| | |
|---|---|
| Micronized | Submicronized |

| | 7.5% w/w | 30% w/w | 7.5% w/w |
|---|---|---|---|
| Time (Min) | alcohol | alcohol | alcohol |
| 15 | 1.01 | 1.22 | 1.10 |
| 30 | 2.07 | 2.36 | 2.12 |
| 60 | 5.21 | 6.40 | 6.38 |
| 120 | 9.19 | 10.03 | 9.86 |
| 180 | 13.85 | 13.78 | 14.30 |
| 240 | 14.38 | 14.64 | 16.36 |

### EXAMPLE 2

This example illustrates the preparation of a pharmaceutical composition of rofecoxib. The pharmaceutical composition is given below in Table 3.

TABLE 3

| Ingredients | Quantity (% w/w) |
|---|---|
| Rofecoxib | 1.0 |
| Carboxypolymethylene (Carbopol 940) | 1.4 |
| Polyethylene glycol (PEG-400) | 12.5 |
| Propylene Glycol | 5.0 |
| Polyethylene glycol-8 | 5.0 |
| glyceryl Caprylate (Labrasol) | |
| Ethanol | 7.5 |
| Triethanolamine | 1.0 |
| Phenoxyethanol | 1.0 |
| Fragrance (Oil of lemon lime) | 0.34 |
| Purified water | to 100 |

Polyethylene glycol, propylene glycol, polyethylene glycol-8 glyceryl caprylate and phenoxyethanol were stirred well to form a dispersion. Rofecoxib (micronized, mean particle size 9.51μ) was then added slowly under continuous stirring. The stirring was continued till a uniform dispersion was formed. Carboxyvinyl polymer was further dispered in the resultant dispersion following which a portion of water was added. Ethanol, fragrance and a solution of triethanolamine was then dispersed. The weight was made upto 500 g with purified water and the resultant mixture was thoroughly agitated until a homogenous composition was obtained. The resultant composition had a pH of 5.76 and a viscosity of 1,70,000 cps.

Similarly, the topical gel formulations were prepared using rofecoxib having a mean particle size of 1.265μ. For the purpose, Rofecoxib (15% w/w) was dispersed in an aqueous solution of polyoxyl 40 hydrogenated caster oil (0.5% w/w) and the resultant suspension was milled to attain submicronized drug particles which were used to formulate the gel preparation by the procedure described above. The resultant composition had a pH of 5.72 and a viscosity of 1,72,000 cps.

The compositions were studied for in vitro release profile using modified Franz diffusion cell and the samples of the receptor media (IPA:Water::80:20) were analyzed for rofecoxib content at prescheduled timings, spectrophotometrically. The results are tabulated in Table 4.

TABLE 4

Flux (μg/ml/cm²)

| Time (Min) | Micronized | Submicronized |
|---|---|---|
| 15 | 3.49 | 3.61 |
| 30 | 8.70 | 9.45 |
| 60 | 10.76 | 12.38 |
| 120 | 13.68 | 15.35 |
| 180 | 15.81 | 17.67 |
| 240 | 16.33 | 18.75 |

### EXAMPLE 3

This example illustrates the preparation of a pharmaceutical composition of rofecoxib without using volatile solubilizing agents. The pharmaceutical composition is given below in Table 5.

TABLE 5

| Ingredients | Quantity (% w/w) |
|---|---|
| Rofecoxib | 1.0 |
| Carboxypolymethylene (Carbopol 940) | 1.4 |
| Polyethylene glycol (PEG-400) | 12.5 |
| Propylene Glycol | 5.0 |
| Polyethylene glycol-8 glyceryl | 5.0 |
| Caprylate (Labrasol) | |
| Triethanolamine | 1.0 |
| Phenoxyethanol | 1.0 |
| Fragrance (Oil of lemon lime) | 0.34 |
| Purified water | to 100 |

Polyethylene glycol, propylene glycol, polyethylene glycol-8 glyceryl caprylate and phenoxyethanol were stirred well to form a dispersion. Rofecoxib was then added slowly under continuous stirring. The stirring was continued till a uniform dispersion was formed. Carboxyvinyl polymer was further dispersed in the resultant dispersion following which a portion of water was added. Fragrance and a solution of triethanolamine was then added. The weight was made upto 500 g with purified water and the resultant mixture was thoroughly agitated until a homogenous composition was obtained. The resultant composition had a pH of 5.83 and a viscosity of 1,56,000 cps.

Rofecoxib was reduced to submicron size range as described in Example 2 and was formulated as topical gel by procedure described above. The resultant composition had a pH of 5.81 and a viscosity of 1,65,000 cps.

The compositions were studied for in vitro release characteristics as described in Example 2. The results are shown in Table 6.

TABLE 6

Flux (μg/ml/cm²)

| Time (Min) | Micronized | Submicronized |
|---|---|---|
| 15 | 3.21 | 3.15 |
| 30 | 8.01 | 8.15 |
| 60 | 9.97 | 10.13 |
| 120 | 12.40 | 12.61 |
| 180 | 14.40 | 14.65 |
| 240 | 15.71 | 15.87 |

The formulations described in Example 1 and 2 were selected for further studies and the evaluation of the anti-inflammatory efficacy and safety of the gel compositions of this invention were substantiated by the following pharmacological experiments.

Evaluation of Efficacy of Gel Formulations in Carrageenan Induced Rat Paw Edema Model of Inflammation

The study was conducted on male Wistar Rats (200±20 g). The anti-inflammatory effect of celecoxib and rofecoxib gel of the present invention was compared with the conventional gel formulations, Nimesulide Transdermal gel 1% w/w (Nimulid Transgel™ Panacea Biotec) and Diclofenac Gel 1% w/w (Voveran Emulgel™, Novartis). Gels were weighed and applied 25 times per minute on the plantar surface of the right hind paw of 6 rats each for the test and saline was applied similarly for the control group. Three hours after the treatment, 0.1 ml of 1% w/v carrageenan in saline was injected sub-plantarly in the treated paw and 0.1 ml of saline was injected in the contralateral paw. Initially and three hours later, paw volume of the hind paws was measured using the plethysmometer and percent swelling was computed as change in paw volume in 3 hours and percent inhibition as the protection observed. The results of the study with different gel formulation at different dose levels are tabulated below.

TABLE 7

| | | | | | ED₅₀ |
|---|---|---|---|---|---|
| | | | | | (mg/kg |
| | | | | | of |
| | | | | | active |
| | Percent inhibition of Edema formation at dose | | | | drug in |
| | (mg/kg) of active drug | | | | applied |
| Study | 1 | 3 | 10 | 30 | gel) |
| Celecoxib gel, | 30.84 ± 14.67 | 42.44 ± 8.29 | 52.65 ± 4.92 | 57.40 ± 8.69 | 7.03 |
| 5% w/w | | | | | |
| (submicronized) | | | | | |
| Celecoxib gel, | 28.74 ± 11.7 | 39.05 ± 14.61 | 50.38 ± 10.51 | 58.31 ± 9.59 | 9.33 |
| 5% w/w | | | | | |
| (micronized) | | | | | |
| Rofecoxib gel, | 34.97 ± 11.31 | 44.28 ± 15.06 | 53.45 ± 4.63 | 62.06 ± 6.1 | 5.86 |
| 1% w/w | | | | | |
| (submicronized) | | | | | |
| Rofecoxib gel | 15.7 ± 11.23 | 36.3 ± 14.69 | 45.5 ± 23.77 | 57.2 ± 18.23 | 10.86 |
| 1% w/w | | | | | |
| (Micronized) | | | | | |
| Diclofenac Gel, | 28.06 ± 11.34 | 41.86 ± 9.63 | 52.16 ± 14.72 | 60.2 ± 7.68 | 6.96 |
| 1% w/w | | | | | |
| (VOVERAN | | | | | |
| EMULGEL) | | | | | |
| Nimesulide | 31.41 ± 12.18 | 40.84 ± 12.27 | 49.08 ± 8.71 | 57.46 ± 10.2 | 9.02 |
| Gel, 1% w/w | | | | | |
| (NIMULID | | | | | |
| TRANSGELI) | | | | | |

The results of the study corroborated that the gel of the present invention were comparable against edema formation with other commercial preparations.

Evaluation of Toxicity of Gel Formulations

The subchronic dermal toxicity was evaluated for gel formulations on Sprague Dawey rats. Groups of 10 rats consisting of 5 males and 5 females each weighing between 210-290 g, used as test animals, were shaved off from the dorsolumbar region, using electric clippers, exposing an area equivalent to 10% of the total body surface. Gel formulations of Rofecoxib 1% w/w (in the dose of 50 mg/kg, 150 mg/kg and 450 mg/kg body weight) and celecoxib 5% w/w (in the dose of 100 mg/kg, 300 mg/kg and 1000 mg/kg body weight) were applied daily for 28 days on the shaven back skin of each test animal and normal saline was applied to the control group treated, similarly. Haematological analysis, biochemical analysis, urine analysis, opthalmological examination, gross pathological examination and histopathological examination did not reveal any abnormality attributable to the treatment. Further, the results for percutaneous lethal dose (LD₅₀) was also extrapolated which are recorded below:

TABLE 8

| | Dose | No. of dead | Percutaneous |
|---|---|---|---|
| | (mg/kg) | animals/No. of | LD₅₀ |
| Formulation | Body weight | animals tested | (mg/kg) |
| Rofecoxib | Control | 0/10 | >450 |
| Gel 1% (w/w) | 50 | 0/10 | |
| | 150 | 0/10 | |
| | 450 | 0/10 | |
| Celecoxib Gel | Control | 0/10 | >1000 |
| 5% (w/w) | 100 | 0/10 | |
| | 300 | 0/10 | |
| | 1000 | 0/10 | |

The result of toxicity studies above is indicative that gel compositions of the present invention are harmless and safe when applied externally.

Evaluation of Efficacy and Tolerability of Topical Gels in Acute Musculoskeletal Pain

The efficacy and tolerability of rofecoxib gel (1%) in the treatment of human patients suffering from extraarticular acute musculoskeletal pain was evaluated in an open, non-comparative and multicentric study of 10 days. The study was conducted in 110 human subjects of either sex aged between 12-76 years and weighing between 35-89 kg, wherein the patients were topically given a total dose of 5 g/day in divided doses (4 times per day), for 10 days. The patients were assessed for efficacy variables as severity of pain, swelling and loss of function using a 4 point scale (0=none, 1=mild, 2=moderate, 3=severe). The results are given in Table 9.

TABLE 9

| | Pain | | Loss of function | | Swelling | |
|---|---|---|---|---|---|---|
| | Pre- | Post- | Pre- | Post- | Pre- | Post- |
| Condition | treatment | treatment | treatment | treatment | treatment | treatment |
| Soft tissue | 2.24 ± 0.60 | 0.59 ± 0.50 | 1.32 ± 0.92 | 0.31 ± 0.46 | 0.97 ± 0.67 | 0.15 ± 0.36 |
| injuries | | | | | | |
| (sprains, | | | | | | |
| strains, | | | | | | |
| contusion) | | | | | | |
| Tendinits | 1.87 ± 0.52 | 0.60 ± 0.51 | 1.07 ± 0.70 | 0.40 ± 0.51 | 0.47 ± 0.52 | 0.00 |
| Epicondylitis of | 2.11 ± 0.30 | 0.22 ± 0.44 | 1.44 ± 0.88 | 0.11 ± 0.33 | 0.56 ± 0.53 | 0.00 |
| elbow | | | | | | |
| Tenosynovitis | 2.14 ± 0.69 | 0.14 ± 0.38 | 0.86 ± 0.69 | 0.14 ± 0.38 | 1.14 ± 0.69 | 0.14 ± 0.38 |
| Humeroscapular | 1.86 ± 0.38 | 1.00 ± 0.00 | 1.29 ± 0.49 | 0.29 ± 0.49 | 0.86 ± 0.69 | 0.00 |
| periarthritis | | | | | | |
| (Ac. Shoulder | | | | | | |
| pain) | | | | | | |
| Other (planter | 2.15 ± 0.38 | 1.00 ± 0.71 | 1.00 ± 0.58 | 0.77 ± 0.60 | 0.62 ± 0.77 | 0.15 + 0.38 |
| fasciitis, low | | | | | | |
| backache, | | | | | | |
| cervical pain, | | | | | | |
| pain knee and | | | | | | |
| ankle) | | | | | | |
| Overall mean | 2.14 ± 0.55 | 0.61 ± 0.54 | 1.23 ± 0.82 | 0.35 ± 0.05 | 0.83 ± 0.68 | 0.11 ± 0.31 |
| scores | | | | | | |
| (n = 110) | | | | | | |

The patients showed clinically and statistically highly significant (p<0.0001) reduction in pain, swelling and improvement in loss of function after treatment with rofecoxib gel 1% (micronized) for 10 days. Moderate to complete recovery was observed in 90% of patients treated with the same.

The patients were also assessed for safety variables as type, severity and frequency of adverse experiences. It was found that only one patient (0.9%) experienced mild erythema at the site of application of rofecoxib gel. No severe or serious adverse event occurred in the study.

As evident rofecoxib gel 1% (micronized) was found to be an effective therapy for the treatment of patients with acute musculoskeletal pain and that the drug was very well tolerated.

Similarly, the efficacy and tolerability of celecoxib gel (5%) in the treatment of extraarticular acute musculoskeletal pain was evaluated in an open, non-comparative and multicentric study of 10 days. The study was conducted in 120 humans subjects of either sex, aged between 18-70 years and weighing between 42-91 kg. The patients were topically administered celecoxib gel (5%) at a dose of 14 g/day in 4 divided doses for 10 days. As mentioned above, the patients were assessed for various parameters of efficacy and tolerability using a 4-point scale and the results are recorded below.

TABLE 10

| | Pain | | Loss of function | | Swelling | |
|---|---|---|---|---|---|---|
| | Pre- | Post- | Pre- | Post- | Pre- | Post- |
| Condition | treatment | treatment | treatment | treatment | treatment | treatment |
| Soft tissue | 2.42 ± 0.54 | 0.54 ± 0.58 | 1.88 ± 0.84 | 0.33 ± 0.56 | 1.25 ± 1.02 | 0.48 ± 0.62 |
| injuries | | | | | | |
| (sprains, | | | | | | |
| strains, | | | | | | |
| contusion) | | | | | | |
| Tendinits | 2.37 ± 0.50 | 0.47 ± 0.51 | 1.89 ± 0.66 | 0.32 ± 0.48 | 0.89 ± 0.94 | 0.21 ± 0.54 |
| Tenosynovitis | 2.17 ± 0.51 | 0.39 ± 0.51 | 1.72 ± 0.83 | 0.22 ± 0.43 | 0.83 ± 0.79 | 0.06 ± 0.24 |
| Epicondylitis of | 2.15 ± 0.38 | 0.54 ± 0.52 | 1.69 ± 0.63 | 0.31 ± 0.48 | 1.00 ± 0.71 | 0.62 ± 0.77 |
| elbow | | | | | | |
| Humeroscapular | 2.63 ± 0.52 | 0.75 ± 0.46 | 2.00 ± 0.76 | 0.63 ± 0.52 | 0.88 ± 0.64 | 0.38 ± 0.74 |
| periarthritis | | | | | | |
| (Ac. Shoulder | | | | | | |
| pain) | | | | | | |
| Other (planter | 2.21 ± 0.58 | 0.07 ± 0.27 | 0.79 ± 0.89 | 0.00 | 0.79 ± 0.80 | 0.21 ± 0.43 |
| fasciitis, | | | | | | |
| myofasciits, | | | | | | |
| low backache, | | | | | | |
| cervical pain, | | | | | | |
| pain knee and | | | | | | |
| ankle) | | | | | | |
| Overall mean | 2.33 ± 0.52 | 0.47 ± 0.53 | 1.72 ± 0.85 | 0.29 ± 0.49 | 1.03 ± 0.90 | 0.35 ± 0.59 |
| scores(n = 120) | | | | | | |

Clinically and statistically highly significant (p<0.0001) reduction in pain, swelling and improvement in loss of reduction was obtained in patients after treatment with celecoxib gel 5% (micronized) for 10 days. Moderate to complete recovery was documented in 97.5% of patients treated with celecoxib gel 5% (micronized).

Likewise, the patients were also evaluated for safety variables and celecoxib gel displayed excellent tolerability. No patients reported any adverse event in this study.

As shown, celecoxib gel 5% is an effective topical therapy for the treatment of patients with acute musculoskeletal pain and that the drug is very well tolerated.

The clinical studies were performed only with topical gels having micronized active ingredient. It may well be understood that as the particle size is reduced, the absorption and thus the efficacy increases and the same may be extrapolated to gel formulations of the present invention wherein the active ingredient used for the formulation development is in the submicron size range. This is also evident from the studies pertaining to evaluation of efficacy of gel formulations in paw edema model of inflammation wherein formulations having reduced particle size showed significantly lower ED₅₀ values for the applied gel.

It may therefore be concluded from the aforementioned studies that the gel compositons of the present invention possess remarkably high percutaneous absorption, medicinal efficacy and safety.

While the invention has been described with an emphasis upon preferred embodiment, it will be obvious to those of ordinary skill in the art that variations in the preferred methods of the present invention may be used and that the invention may be practiced otherwise than as specifically described herein. Accordingly, this invention includes all modifications encompassed within the spirit and scope of the invention as defined by the following claims.

1. A pharmaceutical composition for topical delivery comprising,
a. a pharmaceutically effective amount of drug(s) that acts selectively as a cyclooxygenase-2 enzyme inhibitor, said drug having a mean particle size of less than about 30μ;
b. a gelling agent; and
c. a solubilizing agent.

2. The composition according to claim 1 wherein the drug is selected from the group consisting of celecoxib, rofecoxib, varecoxib, parecoxib, valdecoxib, etodolac, nimesulide, meloxicam and combinations thereof.

3. The composition according to claim 2 wherein the drug is celecoxib.

4. The composition according to claim 2 wherein the drug is rofecoxib.

5. The composition according to claim 1 wherein the drug is present in an amount from about 0.1% to about 25% by weight of the total weight of said composition.

6. The composition according to claim 1 wherein the drug has a mean particle size of less than about 5μ.

7. The composition according to claim 1 wherein the drug has a mean particle size of less than about 0.9μ.

8. The composition according to claim 1 wherein the gelling agent comprises a cellulose ether, vinyl alcohol, vinyl pyrrolidone, natural gum, acrylic polymer, polyoxyethylene-polyoxypropylene copolymer and mixtures thereof.

9. The composition according to claim 8 wherein the cellulose ether is selected from the group consisting of hydroxypropyl cellulose, hydroxyethyl cellulose, hydroxyethyl methylcellulose, methyl cellulose, hydroxypropyl ethylcellulose, hydroxypropyl methylcellulose, carboxymethyl cellulose, sodium carboxymethyl cellulose, hydroxycellulose, derivatives and mixtures thereof.

10. The composition according to claim 8 wherein the vinyl alcohol is polyvinyl alcohol.

11. The composition according to claim 8 wherein the vinyl pyrrolidone is polyvinylpyrrolidones.

12. The composition according to claim 8 wherein the natural gum is selected from the group consisting of karaya gum, locust bean gum, guar gum, gelan gum, xanthan gum, gum arabic, tragacanth carrageenan, pectin, agar, alginic acid, sodium alginate and mixtures thereof.

13. The composition according to claim 8 wherein the acrylic polymer is selected from the group consisting of methacrylates, polyacrylates copolymers and mixtures thereof.

14. The composition according to claim 8 wherein polyoxyethylene-polyoxypropylene copolymer is poloxamer.

15. The composition according to claim 1 wherein the gelling agent comprises about 0.3% to about 40% by weight of the total weight of said composition.

16. The composition according to claim 15 wherein the gelling agent comprises about 0.5% to about 30% by weight of the total weight of said composition.

17. The composition according to claim 1 wherein the solubilizing agent comprises a volatile agent, non-volatile agent and mixtures thereof.

18. The composition according to claim 17 wherein the volatile solubilizing agent is selected from the group consisting of ethanol, denatured ethanol, propanol, isopropanol, butanol and mixtures thereof.

19. The composition according to claim 17 wherein the non-volatile solubilizing agent comprises a glycol and derivatives thereof, polysorbate, sorbitan ester, polyoxyl oil derivatives and mixtures thereof.

20. The composition according to claim 19 wherein the glycol is selected from the group consisting of butylene glycol, propylene glycol, polypropylene glycol, polyethylene glycol, hexylene glycol, polyethylene glycol dodecyl ether, diethylene glycol monoethyl ether, polyethylene glycol-8 glyceryl caprylate, propylene glycol monocaprylate and mixtures thereof.

21. The composition according to claim 19 wherein the polysorbate is selected from the group consisting of polyoxyethylene sorbitan monolaurate, polyoxyethylene sorbitan monopalmitate, polyoxyethylene sorbitan monostearate, polyoxyethylene sorbitan monooleate, polyoxyethylene sorbitan trioleate and mixtures thereof.

22. The composition according to claim 19 wherein the sorbitan ester is selected from the group consisting of sorbitan monolaurate, sorbitan monopalmitate, sorbitan, monostearate, sorbitan monooleate, sorbitan sesquioleate, sorbitan trioleate and mixtures thereof.

23. The composition according to claim 19 wherein the polyoxyl oil derivative is selected from the group consisting of polyoxyl castor oil, polyoxyl 35 castor oil, polyoxyl 40 hydrogenated castor oil, polyoxyl 60 hydrogenated castor oil and mixtures thereof.

24. The composition according to claim 1 wherein the solubilizing agent comprises about 2% to about 60% by weight of the said composition.

25. The composition according to claim 24 wherein the solubilizing agent comprises about 10% to about 40% by weight of the total weight of said composition.

26. The composition according to claim 1 wherein the composition further comprises a pH modifying agent and other pharmaceutically acceptable adjuvants.

27. The composition according to claim 26 wherein the pH modifying agent is an inorganic basic salt or an organic basic salt.

28. The composition according to claim 27 wherein the inorganic basic salt is selected from the group consisting of ammonium hydroxide, magnesium oxide, magnesium hydroxide, calcium hydroxide, sodium hydroxide, potassium hydroxide, lithium hydroxide, aluminium hydroxide, potassium carbonate, sodium bicarbonate and mixtures thereof.

29. The composition according to claim 27 wherein the organic basic salt is an alkanolamine or alkylamine.

30. The composition according to claim 29, wherein the alkanolamine is selected from the group consisting of methanolamine, ethanolamine, propanolamine, butanolamine, dimethanolamine, dibutanolamine, trimethanolamine, triethanolamine, tripropanolamine, diisopropanolamine, tributanolamine, aminomethyl propanol, N-methyl glucamine, tetrahydroxy propylethylene diamine and mixtures thereof.

31. The composition according to claim 29 wherein the alkylamine is selected from the group consisting of methylamine, ethylamine, propylamine, butylamine, diethylamine, dipropylamine, isopropylamine and mixtures thereof.

32. The composition according to claim 26 wherein the composition has a pH of between 2.0 and 8.0

33. The composition according to claim 26 wherein the pharmaceutically acceptable adjuvants comprises penetration enhancers, humectants and/or moisturizers and preservatives.

34. The composition according to claim 33 wherein the penetration enhancer is a terpene, terpene alcohol, essential oils and surfactants.

35. The composition according to claim 34 wherein the penetration enhancer is selected from the group consisting of d-limonene, terpinen-4-ol, menthone, 1,8-cineole, 1-pinene, α-terpineol, carveol, carvone, pulegone, eucalyptol, peppermint oil, sorbitan esters, polysorbates, sodium lauryl sulphate and mixtures thereof.

36. The composition according to claim 33 wherein the humectant and/or moisturizer is selected from the group consisting of sorbitol, glycerin, hexanetriol, butanediol, mannitol, glucose, ethylene glycol, propylene glycol and mixtures thereof.

37. The composition according to claim 33 wherein the preservative is selected from the group consisting of methylparaben, propylparaben, phenoxyethanol, benzyl alcohol, bromopol, chlorocresol, thiomersal, benzalkonium chloride and mixtures thereof.

38. The composition according to claim 1 wherein the composition further comprises opacifiers, fragrances, colour additives, counter-irritants or mixtures thereof.

39. The composition according to claim 1 wherein the composition provides significant protection of the carrageenan induced paw edema formation at an effective dose (ED₅₀) of not more than 15 mg/kg.

40. The composition according to claim 1 comprising celecoxib that provides significant protection of the carrageenan induced paw edema formation at an effective dose (ED₅₀) of about 7.5 mg/kg.

41. The composition according to claim 1 comprising rofecoxib that provides significant protection of the carrageenan induced paw edema formation at an effective dose (ED₅₀) of about 6 mg/kg.

42. The composition according to claim 1 wherein the composition has a viscosity of between 25,000 and 4,00,000 centipoises.

43. The composition according to claim 1 wherein the composition is a gel, a spray, an aerosol, a lotion, a cream or an ointment.
